(19) United States
(12) Patent Application Publication
(10) Pub. No.: US 2010/0069627 A1
(43) Pub. Date: Mar. 18, 2010

(54) NOVEL METHOD FOR THE DIASTEREOSELECTIVE PRODUCTION OF A CHIRAL PRIMARY AMINE ON A STEROID

(75) Inventors: Gilles ODDON, Lyon (FR); Daniel BERNARD, Paris (FR); Gerard CAZENAVE, Montanay (FR); Andre SIMONNET, Saint Didier de Formans (FR); Joelle BOUSQUET-FRANCES, Villeurbanne (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/483,365

(22) Filed: Jun. 12, 2009

Related U.S. Application Data
(63) Continuation of application No. PCT/FR2007/002035, filed on Dec. 11, 2007.

(30) Foreign Application Priority Data
Dec. 13, 2006 (FR) 06 10853

(51) Int. Cl. C07J 41/00; C07J 71/00

(52) U.S. Cl. 540/22; 552/521; 552/522; 552/515

(57) ABSTRACT

The invention relates to a diastereoselective method for obtaining a primary amine on a steroid, comprising the reduction of an oxime by lithium in ammonia at a low temperature in an ether/alcohol mixture.

The present invention relates to a process for diastereoselectively obtaining a primary amine on a steroid.

The process according to the invention is particularly advantageous since it allows the development of novel synthetic pathways for steroids including the diastereoselective obtaining of chiral amines, on an industrial scale.

Synthetic pathways which allow a primary amine to be introduced onto a steroid are described. They generally involve a reduction of substituted or unsubstituted oximes through the action of reducing agents such as hydrides, zinc in acetic acid, or sodium in an alcohol. These processes most commonly produce mixtures in various proportions of alpha- and beta-isomers of the amine. The diastereoisomers must very commonly be isolated by preparative chromatography. Thus, the synthetic pathways do not allow transposition to the industrial level.

Among the prior art documents, mention may more particularly be made of application WO 01/83512, which describes the preparation of steroids bearing an amino group, obtained under stereoselective conditions, by reduction of an azido group of appropriate configuration, using a hydride or hydrogen in the presence of a palladium catalyst.

The transposition to the industrial scale of such techniques, in particular that using an azido intermediate, is very difficult, or even impossible, according to the industrial sites. It is in fact known that the use of azide chemistry on the industrial scale requires the construction of specific plants. In the presence of traces of acids, azides produce hydrazoic acid (or hydrogen azide) which is a highly toxic and highly explosive gas. Heavy metal azides are also highly explosive, and all contact of the azides with alloys containing heavy metals must therefore be avoided. This therefore implies suitable and laborious safety measures for carrying out reactions with sodium azide.

The publication J. Chem. Research (5) 2003, 234-235 recalls that 3-aminosteroids have been obtained from 3-hydroxy derivatives by tosylation, formation of azides and reduction to amines. It adds that this method results in an inversion of the configuration in the 3-position. It describes an alternative 4-stage method which does not bring about any configurational inversion, consisting essentially in treating a ketone in the 3-position with hydroxylamine in pyridine, in reducing the oxime with sodium in isopropanol and in treating the reaction medium with acetic acid.

The publication Bull. Soc. Chim. 1971, n^{o} 11 p. 4072-4078 recalls the various methods for obtaining 3-aminosteroids known at the time, consisting in reducing 3-oximino compounds (Na-alcohol, LiAlH₄-ether/dioxane, LiAlH₄—AlCl₃ complex) and specifies that these methods give rise to primary amines only with difficulty, due to the difficulties in separating the derived compound from the complex mixtures obtained. Mixtures of 3-position isomers and also degradation/transposition compounds are obtained. Two other more stereospecific methods for reducing 3-oximino compounds are described, catalytic hydrogenation in the presence of Adams's platinum, and the action of lithium in ethylamine. The latter produces a mixture of predominantly β-amines, from which it is therefore necessary to separate the α-isomer, by crystallization or chromatography.

The applicant has developed a novel completely stereoselective process for preparing steroidal primary amines starting from oximes, which allows ready transposition to the industrial scale and can be applied generally, provided that the molecule does not otherwise comprise a substitution sensitive to the reaction conditions.

The subject of the present invention is thus a process for the stereoselective preparation of steroid primary amines of α- or β-configuration, at position 1, 2, 3, 4, 6, 7, 11, 12, 15, 16 or 17 on the steroid backbone, characterized in that an oxime of formula (II):

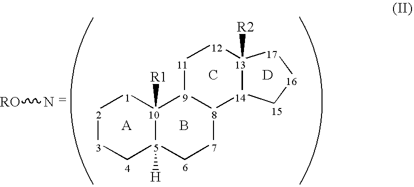

in which R represents a hydrogen atom, a linear, branched or cyclic alkyl radical containing from 1 to 12 carbon atoms, or an aryl or aralkyl radical containing up to 12 carbon atoms, R1 represents a hydrogen atom or a lower alkyl radical containing from 1 to 4 carbon atoms, R2 represents a lower alkyl radical containing from 1 to 4 carbon atoms, the oxime function is located at position 1, 2, 3, 4, 6, 7, 11, 12, 15, 16 or 17 on the backbone, which may be otherwise substituted with one or more groups not sensitive to the reaction conditions defined hereinafter,
is treated with lithium metal in liquid ammonia, at a temperature of between −33° C. and −90° C., in a mixture of a solvent of ether type and of an aliphatic alcohol, and the expected compound of formula (I):

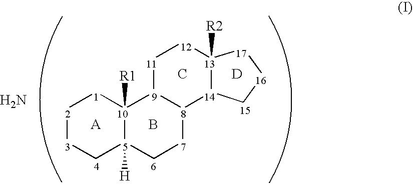

in which the amine of α- or β-configuration is in the position corresponding to that of the oxime on the compound of formula (II), is obtained.

The amine obtained is in the equatorial configuration, which corresponds to the most thermodynamically stable position.

The groups sensitive to the reaction conditions to which reference is made above are well-known to organic chemists, but, as indicated, the process according to the invention can be applied generally and, due to the reactivity of the oxime under the reducing conditions employed, the use of a given amount of lithium, with the reaction being monitored and interrupted when the oxime has disappeared, makes it possible to prevent other groups reputed to be sensitive, such as ester, amide or ketone groups, or even aromatic substituents, or double bonds, being affected.

The groups which cannot be present are essentially conjugated enones.

The amount of lithium used is at least the minimum theoretical amount of 4 equivalents, but, as known by those skilled in the art, a larger amount may be necessary, in particular if the molecule contains one or more labile protons, resulting in consumption of lithium.

A subject of the invention is in particular a process as defined above, characterized in that it is carried out at a temperature of between −50° C. and −80° C.

A subject of the invention is in particular a process as defined above, characterized in that the alcohol used is a linear, branched or cyclic alkanol containing from 1 to 6 carbon atoms, optionally substituted with one or more fluorine atoms.

A subject of the invention is more particularly a process as defined above, characterized in that the alcohol used is a linear or branched alkanol containing from 1 to 4 carbon atoms, optionally substituted with one or more fluorine atoms.

A subject of the invention is in particular a process as defined above, characterized in that the solvent of ether type used is tetrahydrofuran or methyltetra-hydrofuran. However, other ethers known to those skilled in the art, which are liquid under the reaction conditions, can be used according to the invention.

R may be any alkyl, aryl or aralkyl radical as defined above, but a subject of the invention is in particular a process characterized in that R represents a methyl, ethyl or benzyl radical.

As indicated above, the process of the invention can be applied generally and a subject of said invention is in particular a process as defined above, characterized in that the steroid backbone involved is substituted with one or more elements chosen from the group consisting of halogen, free or protected ketone, hydroxyl in free or etherified faun, amino, carboxyl, esterified carboxyl, imide, amide, and a saturated or unsaturated, linear, branched or cyclic, monovalent or divalent carbon chain containing up to 15 carbon atoms, where appropriate interrupted with 1 to 3 oxygen, sulphur or nitrogen atoms, and optionally substituted with hydroxyl or ketone which may be free or protected, halogen, carboxyl or esterified carboxyl, and comprises, where appropriate, one or more double bonds in the A and/or B and/or C and/or D rings, which may or may not be conjugated.

When the steroid backbone is substituted with several elements chosen from the group defined above, this may involve the same element several times, for example halogen, or ketone or hydroxyl which may be free or protected.

The term “halogen” is intended to mean preferably fluorine.

The term “etherified hydroxyl” is intended to mean all usual protections known to chemists, whether it involves the protection of one hydroxyl group or the protection of two hydroxyl groups attached to two adjacent carbons of the backbone.

Mention may, for example, be made of cleavable ethers such as those formed with a (C₁-C₆)alkyl group, in particular methyl or t-butyl, with a (C₁-C₆)alkylphenyl group, in particular benzyl, p-methoxybenzyl, p-nitrobenzyl, allyl ethers, trityl, methoxymethyl, methoxyethoxymethyl, ethoxyethyl, tetrahydropyranyl, silylated ethers, in particular trimethyl, triethyl or triisopropylsilyl ethers, or t-butyldimethyl-silyl or dimethylarylsilyl ethers.

Mention may also be made of cleavable esters such as those formed with an acetyl, benzoyl, phenylacetyl or formyl group or a haloacetyl group such as chloroacetyl, dichloroacetyl, trichloroacetyl or trifluoroacetyl.

Mention may also be made of carbonates, and also of cyclic ketals such as

—O—(CH₂)ₘ—O—, —O—(CH₂)ₘ—S—, —S—(CH₂)ₘ—S— or —O—CH₂—C(C₁-C₄alkyl)₂-CH₂—O—, or else acyclic ketals such as —(CH₃O)₂— or -(EtO)₂—, m preferably being 1, 2 or 3.

The expression “protection of the ketone group” is intended to mean any protection known to chemists, and in particular the ketals and thioketals mentioned above.

The term “amino” is intended to mean primary, secondary or tertiary amino, in particular (C₁-C₆)alkyl- or dialkylamino.

The term “esterified carboxyl” is intended to mean in particular a (C₁-C₆)alkyl ester.

The carbon chain may be any chain known in the steroid field, in particular linear, branched or cyclic alkyl, alkenyl, alkynyl or alkylene, interrupted with 1 to 3 heteroatoms and/or substituted as indicated above.

As an example of a linear or branched alkyl chain containing from 1 to 12 carbon atoms, mention may in particular be made of methyl, ethyl, propyl, butyl, pentyl, hexyl, octyl and their branched isomers such as isopropyl, isobutyl, isopentyl, neopentyl, isohexyl, 3-methylpentyl, sec-butyl, tert-butyl or tert-pentyl.

As an example of a cyclic alkyl chain, mention may in particular be made of cyclopropyl, cyclobutyl, cyclopentyl, cyclohexyl, cycloheptyl or cyclooctyl, optionally substituted, for example, with an alkyl group containing 1 to 4 carbon atoms.

As an example of an alkenyl chain, mention may in particular be made of vinyl, allyl or butenyl.

As an example of an alkynyl chain, mention may in particular be made of ethynyl or propargyl.

As an example of an alkylene chain, mention may in particular be made of methylene or any divalent chain derived from the above alkyls. This chain may, where appropriate, be attached to two adjacent carbons of the backbone and may also form a bicyclic system.

When the rings contain one or more double bonds, the latter are in particular at position 1(2), 3(4), 1, 3, 5, 5(6), 6(7), 9(11), 15(16) or 16(17).

A subject of the invention is in particular a process as defined above, characterized in that, when the steroid is substituted with an alkylene chain, the latter is not a methylene in the 17-position.

A subject of the invention is more particularly a process as defined above, characterized in that the steroid backbone involved is substituted with one or more elements chosen from the group consisting of fluorine, free or protected ketone, free or protected hydroxyl, amino, ether, amide, imide, and alkyl, alkenyl, alkynyl and alkylene chains as defined above, and comprises, where appropriate, one or more double bonds in the A and/or B and/or C and/or D rings, which may or may not be conjugated.

A subject of the invention is most particularly a process as defined above, characterized in that the steroid backbone involved is substituted with one or more elements chosen from the group consisting of free or protected ketone, free or protected hydroxyl, and a linear or branched alkyl chain containing up to 12 carbon atoms, and comprises, where appropriate, one or two double bonds in the A and/or B and/or C and/or D rings.

The present invention is illustrated by the examples which follow:

### EXAMPLE 1

### Synthesis of 3β-amino stanolone

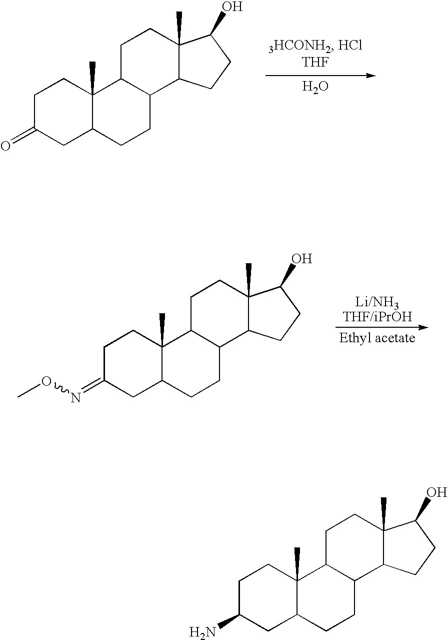

Preparation of stanolone 3-Z,E-methyloxime

30 g of stanolone are dissolved in 120 ml of tetrahydrofuran, at ambient temperature. 21.54 ml of triethylamine (1.5 eq) and 18.8 ml of aqueous sodium hydroxide at 32% m/m (2 eq) are added to this solution, at 20° C. 47.6 ml of methylhydroxylamine hydrochloride in aqueous solution at 31% m/m (1.9 eq) are added with vigorous stirring. The two-phase solution is stirred for 3 h at 20° C. and then for 2 h at 60° C. and the end of the reaction is verified by TLC. The mixture is vacuum-distilled at 60° C., the reaction volume being kept constant through the addition of water, until the refractive index of the distillate is that of water.

The product crystallizes during the solvent exchange. The suspension is stirred for 16 h at 20° C. and then 180 ml of water are added to the suspension, at 20° C. The mixture is stirred for 30 minutes at this temperature, and then the solid is spin-filter-dried and washed with water. The product is dried at 40° C. for 18 h.

33.3 g of stanolone 3-Z,E-methyloxime are obtained (yield=100%).

NMR spectrum: 1H at 300 MHz, DMSO d6 referenced at 2.52 ppm. δ in ppm. 50/50 mixture of Z and E isomers.

| | Multiplicity |
|---|---|
| δ ¹H obs. (ppm) | J (in Hz) |
| 0.59-0.70 | m, 1H |
| 0.65 | s, 3H |
| 0.75-1.43 | m, 11.5H |
| 0.86 | s, 3H |
| 1.43-2.08 | M, 8H |
| 2.16 | m, 0.5H |
| 2.76 | dd, 0.5H (J = 15.4 and 3.4) |
| 2.98 | m, 0.5H |
| 3.44 | m, 1H |
| 3.69 | broad S, 3H |
| 4.41 | d, (1H J = 4.8) |

Mass spectrum: Electrospray ionization in positive mode. MH+ = (m/z) = 320

3β-aminostanolone

20 g of stanolone 3-Z,E-methyloxime are suspended in 200 ml of tetrahydrofuran and 25 ml of isopropanol. The suspension is added to 100 ml of liquid ammonia at −50° C. The mixture is cooled to −70° C. and 1.942 g of lithium granules (4.47 eq) are added in 9 fractions. The mixture is stirred for 1 hour, and then the reaction is treated by adding 16.7 g of ammonium chloride (5 eq). The suspension is then left to come back up to ambient temperature, 40 ml of water are added at 20° C., and then the mixture is vacuum-distilled at 60° C., the reaction volume being kept constant through the regular addition of water, until the refractive index of the distillate is close to that of water. The aqueous suspension is cooled to 20° C. and extracted with 200 ml of methylene chloride. An insoluble material is filtered off and the organic phase is evaporated to dryness. 6.95 g of dry extract are obtained.

In addition, the insoluble material is taken up in 700 ml of methylene chloride and 550 ml of water and the pH is adjusted to 12.5 by adding 2 ml of aqueous sodium hydroxide at 32% m/m. The organic phase is evaporated to dryness and a further 10.4 g of product are obtained. The two products are combined, i.e. 17.35 g, yield=95.1%.

5 g of this product are suspended in 150 ml of ethyl acetate for 1 h at 20° C., and the product is filtered off and washed with ethyl acetate.

2.5 g of pure 3β-aminostanolone are obtained.

NMR spectrum: 1H at 400 MHz, CDCl₃ d1 referenced at 7.27 ppm.

| | Multiplicity |
|---|---|
| δ ¹H obs. (ppm) | J (in Hz) |
| 0.64 | m, 1H |
| 0.73 | s, 3H |
| 0.80 | s, 3H |
| 0.87 | m, 1H |
| 0.92-1.15 | m, 5H |
| 1.17-1.32 | m, 5H |
| 1.32-1.53 | m, 3H |
| 1.53-1.62 | m, 2H |
| 1.63-1.73 | m, 3H |
| 1.79 | dt, 1H (J = 12.6 and 3.2) |
| 2.05 | m, 1H |
| 2.64 | m, 1H |
| 3.62 | t, 1H (J = 8.0) |

α/β ratio of the primary amine = 0/100
Mass spectrum: Electrospray ionization in positive mode: MH+ (m/z) = 292.

### EXAMPLE 2

### 17β-amino-DHEA

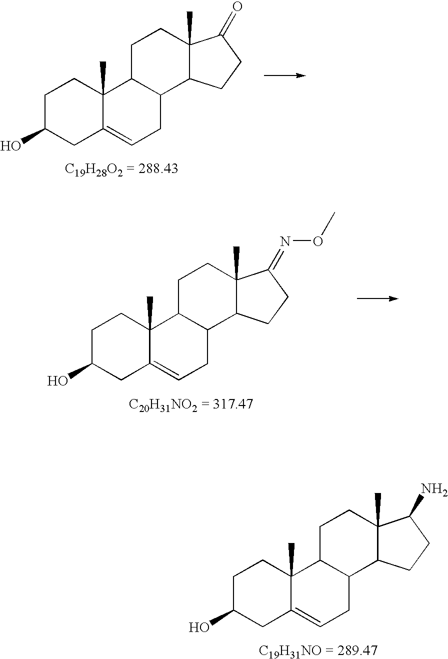

### Preparation of DHEA 17-E-methyloxime

50 g of DHEA are dissolved in 200 ml of toluene and 124 ml of pyridine (8.9 eq). 16.25 g of methoxylamine hydrochloride (98% purity, i.e. 1.1 eq.) are added at 20° C. The mixture is stirred for 5 h at 50° C. and then for 48 h at 30° C. The solid is spin-filter-dried and washed with water, and then dried under vacuum.

In addition, the filtrate is separated by settling out and the organic phase is washed with two times 500 ml of water. The organic phase is vacuum-distilled to dryness. The dry extract and the crystals previously filtered off are mixed together. 53.07 g of DHEA 17-E-methyloxime are obtained, i.e. a total yield of 96.4%.

NMR spectrum: 400 MHz, CDCl₃ d1.

| | δ ¹H | Multiplicity | δ ¹³C |
|---|---|---|---|
| Number | obs. | J (in Hz) | obs. |
| 1 | 1.10 | dd (J = 4.0-13.0) | 37.7 |
| | 1.87 | m | |
| 2 | 1.52 | m | 32.2 |
| | 1.83 | m | |
| 3 | 3.54 | tt (4.5-11.0) | 72.0 |
| 4 | 2.26 | ddd (1.5-11.0-13.0) | 42.7 |
| | 2.32 | ddd (1.5-4.5-13.0) | |
| 5 | — | — | 141.3 |
| 6 | 5.37 | d (5.5) | 121.5 |
| 7 | 1.64 | m | 31.9 |
| | 2.06 | ddd (2.5-5.5-12.0) | |
| 8 | 1.61 | | 31.1 |
| 9 | 1.02 | m | 50.7 |
| 10 | — | — | 37.0 |
| 11 | 1.54 | m | 21.2 |
| | 1.66 | m | |
| 12 | 1.45 | m | 34.4 |
| | 2.00 | dm (12.0) | |
| 13 | — | — | 44.1 |
| 14 | 1.17 | ddd (6.0-10.5-13.0) | 54.6 |
| 15 | 1.36 | m | 24.0 |
| | 1.81 | m | |
| 16 | 2.40 | dd (8.5-19.0) | 26.4 |
| | 2.50 | ddd (1.5-9.0-19.0) | |
| 17 | — | — | 170.9 |
| 18-Me | 0.93 | s | 17.5 |
| 19-Me | 1.04 | s | 19.9 |
| OMe | 3.83 | s | 61.8 |

The proton in the 3-position is axial (3 beta-alcohol).

| Number | δ ¹H obs. | δ ¹H lit ΔE | δ ^{1H}lit ΔZ | δ ¹³Cobs. | δ ¹³C lit ΔE | δ ¹³C lit ΔZ |
|---|---|---|---|---|---|---|
| 18-Me | 0.93 | 0.93 | 1.05 | 17.5 | 16.9 | 13.4 |
| 17 | — | — | — | 170.9 | 170.0 | 170.4 |
| 16 | 2.40-2.50 | 2.53-2.52 | 2.42-2.32 | 26.4 | 25.0 | 29.2 |

Obs = observed,
lit = according to the literature
Mass spectrum: Electrospray ionization in positive mode. MH+ = 318.

17β-amino-DHEA

50 ml of ammonia are condensed at −50° C. and a solution of 5 g of DHEA 17-E-methyloxime in 50 ml of tetrahydrofuran and 7.9 ml of isopropanol (i.e. 6.55 eq) is added at this temperature. 1.078 g of lithium granules (i.e. 9.86 eq) are added at −50° C., fractionwise and under argon. The mixture is stirred for 8 hours at −50° C. and then the temperature is allowed to return to 20° C. The product crystallizes as the temperature comes back up. 50 ml of water are added and the suspension is then stirred for 2 h at 20° C. and then the product is spin-filter-dried and washed with water. The solid is vacuum-dried at 40° C. and 4.55 g of 17β-amino-DHEA are obtained, i.e. a yield of 100%.

NMR spectrum: 400 MHz, CDCl₃ d1

| | | Multiplicity | δ ¹³C |
|---|---|---|---|
| Number | δ ¹H obs. | J (in Hz) | obs. |
| 1 | 1.05 | m | 38.5 |
| | 1.87 | dt (13.5-3.5) | |
| 2 | 1.48 | m | 32.2 |
| | 1.78 | m | |
| 3 | 3.39 | tt (5.5-11.0) | 72.2 |
| 4 | 2.17 | m | 43.0 |
| | 2.22 | m | |
| 5 | — | — | 142.1 |
| 6 | 5.34 | d (5.0) | 122.1 |
| 7 | 1.55 | m | 32.7 |
| | 1.99 | m | |
| 8 | 1.49 | m | 33.5 |
| 9 | 0.95 | m | 51.8 |
| 10 | — | — | 37.6 |
| 11 | 1.48 | m | 21.8 |
| | 1.61 | m | |
| 12 | 1.01 | m | 37.6 |
| | 1.83 | dt (13.0-3.5) | |
| 13 | — | — | 43.3 |
| 14 | 0.98 | m | 54.6 |
| 15 | 1.21 | dq (5.5-12.0) | 24.6 |
| | 1.64 | m | |
| 16 | 1.32 | ddt (2.5-9.0-12.0) | 31.5 |
| | 1.99 | m | |
| 17 | 2.60 | t (9.0) | 63.4 |
| 18-Me | 0.69 | s | 11.2 |
| 19-Me | 1.03 | s | 19.9 |

The proton in the 3-position is alpha, therefore 3β-alcohol.
The 17beta-amino product is 96% pure.
α/β of the primary amine = 4/96.
Mass spectrum: Electrospray ionization in positive mode MH⁺ = 290.

### EXAMPLE 12β-amino hecogenin

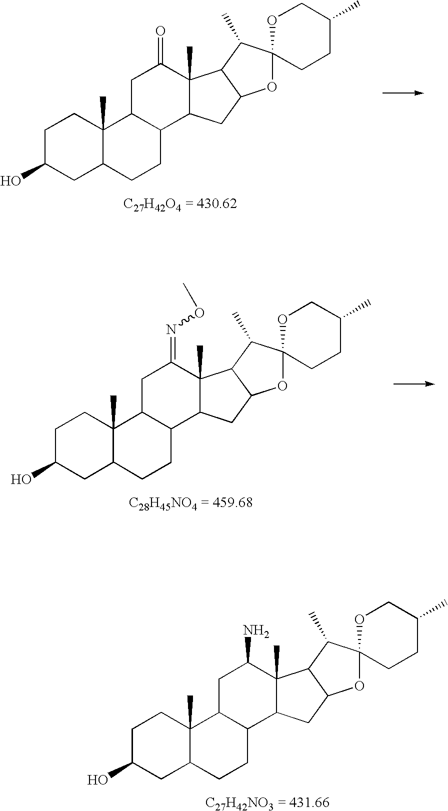

Preparation of 12-Z,E-methyloxime hecogenin
20 g of hecogenin are dissolved, at 20° C., in 40 ml of toluene and 15.8 ml of pyridine (i.e. 8.9 eq). 2.03 g of methoxylamine hydrochloride (i.e. 1.1 eq) are added at 20° C. A white suspension is obtained, which is stirred for 3 h at 50° C. and 15 h at 30° C. The product is spin-filter-dried and washed with water. The product is vacuum-dried at 40° C. 7.05 g of 12-Z,E-methyloxime hecogenin are obtained, i.e. a yield of 69.5%.

NMR spectrum: 400 MHz, CDCl₃ d1.

### Proton Spectrum:

| | | Multiplicity |
|---|---|---|
| Number | δ ¹H obs. | J (in Hz) |
| 1 | 1.07 | M |
| | 1.76 | M |
| 2 | 1.45 | M |
| | 1.85 | M |
| 3 | 3.60 | tt (5.0 and 11.0) |
| 4 | 1.32 | M |
| | 1.58 | M |
| 5 | 1.13 | M |
| 6 | 1.61 | m |
| | 1.72 | m |
| 7 | 0.91 | m |
| | 1.75 | m |
| 8 | 1.76 | m |
| 9 | 0.87 | m |
| 10 | — | — |
| 11 | 1.65 | m |
| | 3.16 | dd (5.0-15.0) |
| 12 | — | — |
| 13 | — | — |
| 14 | 1.35 | m |
| 15 | 1.40 | m |
| | 2.07 | m |
| 16 | 4.39 | m |
| 17 | 2.56 | dd (6.5-8.5) |
| 18-Me | 0.95 | s |
| 19-Me | 0.88 | s |
| 20 | 1.86 | m |
| 21 | 1.10 | d (7.0) |
| 22 | — | — |
| 23 | 1.34 | m |
| 24 | 1.47-1.65 | m-m |
| 25 | 1.67 | m |
| 26 | 3.38 | t (11.0) |
| | 3.50 | ddd (1.5-4.0-11.0) |
| 27-Me | 0.80 | d |
| OMe | 3.78 | s |

### Carbon Spectrum:

| Number | δ ¹³C obs. |
|---|---|
| 1 | 37.0 |
| 2 | 31.8 |
| 3 | 71.3 |
| 4 | 38.5 |
| 5 | 45.2 |
| 6 | 32.1 |
| 7 | 32.4 |
| 8 | 32.7 |
| 9 | 54.0 |
| 10 | 36.4 |
| 11 | 21.0 |
| 12 | 163.8 |
| 13 | 47.6 |
| 14 | 56.7 |
| 15 | 31.3 |
| 16 | 80.2 |
| 17 | 56.3 |
| 18-Me | 17.4 |
| 19-Me | 12.3 |
| 20 | 42.7 |
| 21 | 13.6 |
| 22 | 109.6 |
| 23 | 28.8 |
| 24 | 29.3 |
| 25 | 30.7 |
| 26 | 67.3 |
| 27-Me | 17.5 |
| OMe | 61.5 |

19-Me: 12.3 ppm (5α). H in 3-position: 3.60 ppm; (it is axial, therefore α, since it emerges in the form of a triplet with J = 5.0 and 11.0 Hz.
Mass spectrum: carried out by electrospray ionization in positive mode. Presence of the two isomers Z and E. MH⁺ = 460.

12β-amino hecogenin

50 ml of ammonia are condensed at −50° C. and a solution of 5 g of 17-Z,E-methyloxime hecogenin in 50 ml of tetrahydrofuran and 5.45 ml of isopropanol (i.e. 6.5 eq) is added at this temperature. 0.393 g of lithium granules (i.e. 5.21 eq) is added with stirring and under an inert gas at −50° C., fractionwise and in 3 hours. The mixture is stirred for . . . hours, and then the temperature is allowed to return to 20° C. 50 ml of water are added and the tetrahydrofuran is vacuum-distilled. 100 ml of water are added and the mixture is stirred for 24 h at 20° C. The product is spin-filter-dried and washed with water. The solid is vacuum-dried at 40° C.

4.65 g of 12beta-amino hecogenin are obtained, i.e. a yield of 99%.

NMR spectrum: 400 MHz, CDCl₃ d1.

### Proton Spectrum:

| | | Multiplicity | |
|---|---|---|---|
| Number | δ ¹H obs. | J (in Hz) | δ ¹³C obs. |
| 1 | 0.98 | dt (4.0-13.0) | 37.3 |
| | 1.72 | m | |
| 2 | 1.40 | m | 32.0 |
| | 1.80 | m | |
| 3 | 3.59 | tt (4.511.0) | 71.5 |
| 4 | 1.29 | m | 38.5 |
| | 1.59 | m | |
| 5 | 1.10 | m | 45.2 |
| 6* | 1.30 | m | 29.0 |
| | 1.30 | m | |
| 7 | 0.88 | m | 32.5 |
| | 1.69 | m | |
| 8 | 1.50 | m | 34.7 |
| 9 | 0.78 | m | 54.0 |
| 10 | — | — | 35.9 |
| 11 | 1.22 | m | 30.4 |
| | 1.74 | m | |
| 12 | 2.58 | dl (10.0) | 61.1 |
| 13 | — | — | 45.3 |
| 14 | 1.09 | m | 56.2 |
| 15 | 1.37 | m | 31.8 |
| | 2.02 | ddd (6.0-7.0-12.0) | |
| 16 | 4.43 | ql (7.0) | 81.0 |
| 17 | 1.88 | m | 61.8 |
| 18-Me | 0.78 | s | 10.9 |
| 19-Me | 0.83 | s | 12.9 |
| 20 | 1.88 | m | 42.4 |
| 21 | 1.08 | d (7.0) | 14.9 |
| 22 | — | — | 109.6 |
| 23* | 1.63 | m | 32.4 |
| | 1.68 | m | |
| 24 | 1.45 | m-m | 29.3 |
| | 1.63 | | |
| 25 | 1.63 | m | 30.6 |
| 26 | 3.36 | t (11.0) | 67.2 |
| | 3.47 | ddd (2.0-4.0-11.0) | |
| 27-Me | 0.80 | d (6.0) | 17.6 |

*attributions that may be inversed.
19-Me: 12.9 ppm, which is characteristic of the 5α series. H in the 3-position: 3.59 ppm; it is axial, therefore α, since it emerges in the form of a triplet, with J = 4.5 and 11.0 Hz.
α/β of the primary amine = 5/95.
Mass spectrum: Electrospray ionization in positive mode MH⁺ = 432.

### EXAMPLE 4

### 6α-aminocholestanol

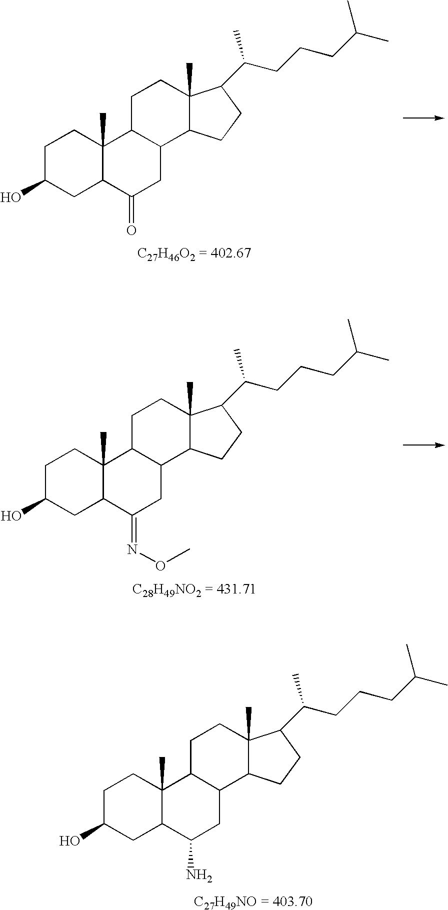

Preparation of 6-E-methyloxime cholestanol

10 g of 6-oxocholestanol (99% purity) are dissolved, at 20° C., in 40 ml of toluene and 17.63 ml of pyridine (i.e. 8.9 eq). 2.30 g of methoxylamine hydrochloride (1.1 eq) are added at 20° C. A white suspension is obtained, which is stirred for 6 h at 50° C. and for 30 h at 25° C. 40 ml of water are added, the mixture is separated by settling out and the aqueous phase is removed. The organic phase is taken up and washed with water. The solution is distilled to dryness. The solid is suspended in 40 ml of n-heptane, spin-filter-dried, and washed with 10 ml of n-heptane. The product is vacuum-dried.

10.14 g of 6-E-methyloxime cholestanol are obtained, i.e. a yield of 95.5%.

NMR spectrum: CDCl₃: ΔE isomer (OMe cis relative to the CH₂ in the 7-position); H in the 3-position axial, OH in the 3-position is therefore β (5α series).

| | | Multiplicity | |
|---|---|---|---|
| Number | δ ¹H obs. | J (in Hz) | δ ¹³C obs. |
| 1 | 1.14 | m | 36.5 |
| | 1.78 | dt (13.0-3.5) | |
| 2 | 1.41 | m | 31.3 |
| | 1.86 | m | |
| 3 | 3.57 | tt (4.5-11.0) | 71.6 |
| 4 | 1.54 | m | 32.2 |
| | 2.00 | m | |
| 5 | 1.93 | dd (2.5-12.0) | 50.2 |
| 6 | — | — | 159.4 |
| 7 | 1.20 | m | 30.7 |
| | 3.22 | dd (4.5-13.5) | |
| 8 | 1.51 | m | 36.3 |
| 9 | 1.13 | m | 57.0 or |
| | | | 56.8 |
| 10 | — | — | 43.2 |
| 11 | 1.32 | m | 21.8 |
| | 1.57 | m | |
| 12 | 1.14 | m | 40.0 |
| | 2.02 | m | |
| 13 | — | — | 49.7 |
| 14 | 0.92 | m | 54.9 |
| 15 | 1.14 | m | 24.4 |
| | 1.62 | m | |
| 16 | 1.25 | m | 28.6 |
| | 1.85 | m | |
| 17 | 1.13 | m | 56.8 or |
| | | | 57.0 |
| 18-Me | 0.66 | s | 12.5 |
| 19-Me | 0.76 | s | 13.0 |
| 20 | 1.40 | m | 36.0 |
| 21 | 0.91 | d (6.5) | 19.0 |
| 22 | 1.04 | m | 36.7 |
| | 1.36 | m | |
| 23 | 1.14 | m | 24.2 |
| | 1.34 | m | |
| 24 | 1.14 | m | 40.0 |
| 25 | 1.54 | m | 28.2 |
| 26 and | 0.86 | d (6.5) | 22.9 |
| 27 | 0.87 | d (6.5) | |
| OMe | 3.82 | s | 61.7 |

Mass spectrum: Electrospray ionization in positive mode. MH⁺ = 432.

### 6α-Aminocholestanol

50 ml of ammonia are condensed at −50° C. and a suspension of 5 g of 6-E-methyloxime cholestanol in 50 ml of tetrahydrofuran and 5.80 ml of isopropanol (i.e. 6.5 eq) is added at this temperature. The mixture is cooled to −65° C. and 0.490 g of lithium granules (i.e. 6.1 eq) is added with stirring, fractionwise, in 5 hours, under argon. The mixture is stirred for 1 hour, and then the temperature is allowed to return to 20° C.

50 ml of water are added and the tetrahydrofuran is distilled off under vacuum. The product is spin-filter-dried and washed with water. The solid is vacuum-dried at 40° C. and a yield of 80.2% of 6alpha-aminocholestanol is obtained.

NMR spectrum: 400 MHz, deuteriated methanol.

| | | Multiplicity | |
|---|---|---|---|
| Number | δ ¹H obs. | J (in Hz) | δ ¹³C obs. |
| 1 | 1.00 | m | 38.3 |
| | 1.72 | m | |
| 2 | 1.41 | m | 31.6 |
| | 1.75 | m | |
| 3 (axial) | 3.48 | tt (4.5-11.0) | 71.7 |
| 4 | 1.16 | m | 33.2 |
| | 1.98 | dt (12.0-3.5) | |
| 5 | 0.91 | m | 52.7 |
| 6 (axial) | 2.55 | dt (3.5-11.0) | 49.9 |
| 7 | 0.77 | ql (12.0) | 42.0 |
| | 1.87 | dt (12.0-3.5) | |
| 8 | 1.49 | m | 35.7 |
| 9 | 0.70 | m | 55.3 |
| 10 | — | — | 36.7 |
| 11 | 1.33 | m | 22.1 |
| | 1.53 | m | |
| 12 | 1.15 | m | 41.1 |
| | 2.02 | dt (12.0-3.5) | |
| 13 | — | — | 43.5 |
| 14 | 1.06 | m | 57.3 |
| 15 | 1.19 | m | 24.9 |
| | 1.40 | m | |
| 16 | 1.30 | m | 29.5 |
| | 1.85 | m | |
| 17 | 1.13 | m | 57.5 |
| 18-Me | 0.69 | s | 12.4 |
| 19-Me | 0.84 | s | 13.7 |
| 20 | 1.39 | m | 36.8 |
| 21 | 0.93 | d (7.0) | 19.0 |
| 22 | 1.03 | m | 37.1 |
| | 1.38 | m | |
| 23 | 1.13 | m | 25.0 |
| | 1.64 | m | |
| 24 | 1.16 | m | 40.7 |
| 25 | 1.53 | m | 29.0 |
| 26 and | 0.87 | d (7.0) | 23.0 |
| 27 | 0.88 | d (7.0) | 23.0 |

H in the 3-position axial, OH in the 3-position is β (5α series).
α/β of the primary amine = 100/0.
Mass spectrum: MH⁺ = 404.

What is claimed is:

1. A process for the stereoselective preparation of steroid primary amines of α- or β-configuration, at position 1, 2, 3, 4, 6, 7, 11, 12, 15, 16 or 17 on the steroid backbone, comprising:
contacting an oxime of formula (II): 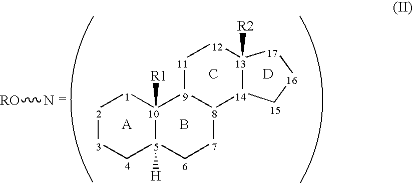
in which R represents a hydrogen atom, a linear, branched or cyclic alkyl radical containing from 1 to 12 carbon atoms, or an aryl or aralkyl radical containing up to 12 carbon atoms, R1 represents a hydrogen atom or a lower alkyl radical containing from 1 to 4 carbon atoms, R2 represents a lower alkyl radical containing from 1 to 4 carbon atoms, the oxime function is located at position 1, 2, 3, 4, 6, 7, 11, 12, 15, 16 or 17 on the backbone, which can otherwise be substituted with one or more groups not sensitive to the reaction conditions defined hereinafter; with lithium metal in liquid ammonia, at a temperature of between −33° C. and −90° C., in a mixture of a solvent of ether type and of an aliphatic alcohol; to obtain an expected compound of formula (I): 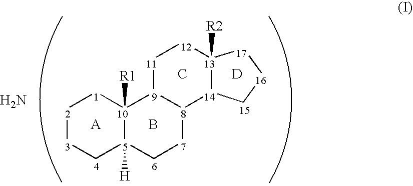
in which the amine of α- or β-configuration is in the position corresponding to that of the oxime on the compound of formula (II).

2. The process according to claim 1, for the stereoselective preparation of steroid primary amines of α- or β-configuration, at position 1, 2, 3, 4, 6, 7, 11, 12, 15, 16 or 17 on the steroid backbone, comprising:
contacting an oxime of formula (II): 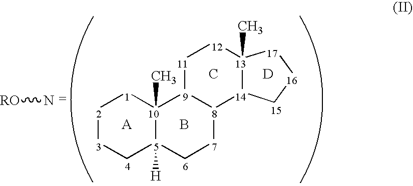
in which R represents a hydrogen atom, a linear, branched or cyclic alkyl radical containing from 1 to 12 carbon atoms, or an aryl or aralkyl radical containing up to 12 carbon atoms, the oxime function is located at position 1, 2, 3, 4, 6, 7, 11, 12, 15, 16 or 17 on the backbone, which can be otherwise substituted with one or more groups not sensitive to the reaction conditions defined hereinafter; with lithium metal in liquid ammonia, at a temperature of between −33° C. and −90° C., in a mixture of a solvent of ether type and of an aliphatic alcohol, to obtain an expected compound of formula (I): 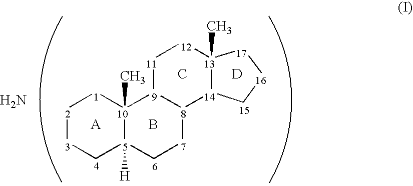
in which the amine of α- or β-configuration is in the position corresponding to that of the oxime on the compound of formula (II).

3. The process according to claim 1, wherein the process is carried out at a temperature of between −50° C. and −80° C.

4. The process according to claim 1, wherein the alcohol used is a linear, branched or cyclic alkanol containing from 1 to 6 carbon atoms, optionally substituted with one or more fluorine atoms.

5. The process according to claim 1, wherein the alcohol used is a linear or branched alkanol containing from 1 to 4 carbon atoms, optionally substituted with one or more fluorine atoms.

6. The process according to claim 1, wherein the solvent of ether type used is tetrahydrofuran or methyltetrahydrofuran.

7. The process according to claim 1, wherein R represents a methyl, ethyl or benzyl radical.

8. The process according to claim 1, wherein the backbone of the steroid of formula (II) involved is substituted with one or more elements chosen from the group consisting of halogen, free or protected ketone, hydroxyl in free or etherified form, amino, carboxyl, esterified carboxyl, imide, and a saturated or unsaturated, linear, branched or cyclic, monovalent or divalent carbon chain containing up to 15 carbon atoms, where appropriate interrupted with 1 to 3 oxygen, sulphur or nitrogen atoms, and optionally substituted with hydroxyl or ketone which may be free or protected, halogen, carboxyl or esterified carboxyl, and comprises, where appropriate, one or more double bonds in the A and/or B and/or C and/or D rings, which may or may not be conjugated.

9. The process according to claim 1, wherein, when the steroid is substituted with an alkylene chain, the alkylene chain is not a methylene in the 17-position.

10. The process according to claim 1, wherein the steroid backbone involved is substituted with one or more elements chosen from the group consisting of fluorine, free or protected ketone, free or protected hydroxyl, amino, ether, amide, imide, and alkyl, alkenyl, alkynyl and alkylene chains as defined above, and comprises, where appropriate, one or more double bonds in the A and/or B and/or C and/or D rings, which may or may not be conjugated.

11. The process according to claim 1, wherein the steroid backbone involved is substituted with one or more elements chosen from the group consisting of free or protected ketone, free or protected hydroxyl, and a linear or branched alkyl chain containing up to 12 carbon atoms, and comprises, where appropriate, one or two double bonds in the A and/or B and/or C and/or D rings.
